(19) United States
(12) Patent Application Publication
(10) Pub. No.: US 2023/0380703 A1
(43) Pub. Date: Nov. 30, 2023

(54) BIOLOGICAL INFORMATION ESTIMATING DEVICE AND BIOLOGICAL INFORMATION ESTIMATING METHOD

(71) Applicant: SHARP KABUSHIKI KAISHA, Sakai City (JP)

(72) Inventors: Ryota TOMIZAWA, Sakai City (JP); Yoshimoto AKAGI, Sakai City (JP); Yoshihisa ADACHI, Sakai City (JP)

(21) Appl. No.: 18/202,603

(22) Filed: May 26, 2023

(30) Foreign Application Priority Data
May 30, 2022 (JP) 2022-087425

(51) Int. Cl. A61B 5/021; A61B 5/024

(52) U.S. Cl. CPC A61B 5/02116; A61B 5/024; A61B 5/7235

(57) ABSTRACT

A biological information estimating device includes: a detection unit configured to detect a pulse wave on a living organism; a modifying unit configured to modify a length of a measurement period based on a pulse pace of the living organism; and an estimating unit configured to estimate biological information from the pulse wave detected in the measurement period.

### FIELD OF THE INVENTION

The present disclosure relates to biological information estimating devices and biological information estimating methods. The present application claims the benefit of priority to Japanese Patent Application, Tokugan, No. 2022-087425 filed on May 30, 2022, the entire contents of which are incorporated herein by reference.

### BACKGROUND

Japanese Unexamined Patent Application Publication, Tokukai, No. 2005-185681 discloses a blood pressure meter.

To use the blood pressure meter, a cuff is wound around an artery passage part of the user. A pulse wave signal is discriminated from the in-cuff pressure detected by a pressure sensor in the process of slowly reducing the pressure of the cuff. A blood pressure value is determined from a pulse wave signal and a pressure value. In personal mode, measurement conditions for measuring the blood pressure of a particular user and blood pressure determining conditions for determining a maximum blood pressure value and a minimum blood pressure value for the particular user are specified on the basis of previous measurements on the particular user. Measurement time can be hence reduced (Abstract).

### SUMMARY

The blood pressure meter disclosed in Japanese Unexamined Patent Application Publication, Tokukai, No. 2005-185681 is capable of reducing measurement time only when measurement conditions and blood pressure determining conditions can be specified on the basis of previous measurements on the particular user. Additionally, to reduce measurement time, the user needs to be identified.

The present disclosure has been made in view of these issues. The present disclosure, in an aspect thereof, has an object to, for example, provide a biological information estimating device and a biological information estimating method both of which can reduce the time required to estimate biological information without having to identifying a living organism.

The present disclosure, in an aspect thereof, is directed to a biological information estimating device including: a detection unit configured to detect a pulse wave on a living organism; a modifying unit configured to modify a length of a measurement period based on a pulse pace of the living organism; and an estimating unit configured to estimate biological information from the pulse wave detected in the measurement period.

The present disclosure, in another aspect thereof, is directed to a biological information estimating method including: a) detecting a pulse wave on a living organism; b) modifying a length of a measurement period based on a pulse pace of the living organism; and c) estimating biological information from the pulse wave detected in the measurement period.

### BRIEF DESCRIPTION OF DRAWINGS

FIG. 1 is a block diagram of a biological information estimating device in accordance with Embodiment 1.

FIG. 2 is a flow chart representing a first example of the flow of a process performed by a modifying unit included in a biological information estimating device in accordance with Embodiment 1.

FIG. 3 is a graph representing an exemplary timing for the modifying unit included in the biological information estimating device in accordance with Embodiment 1 to end a measurement period when the pulse pace of a living organism is slow.

FIG. 4 is a graph representing an exemplary timing for the modifying unit included in the biological information estimating device in accordance with Embodiment 1 to end a measurement period when the pulse pace of a living organism is fast.

FIG. 5 is a graph representing a first example of local maximum point selection performed by the modifying unit included in the biological information estimating device in accordance with Embodiment 1.

FIG. 6 is a graph representing a first example of local minimum point selection performed by the modifying unit included in the biological information estimating device in accordance with Embodiment 1.

FIG. 7 is a graph representing a second example of local maximum point selection performed by the modifying unit included in the biological information estimating device in accordance with Embodiment 1.

FIG. 8 is a graph representing a second example of local minimum point selection performed by the modifying unit included in the biological information estimating device in accordance with Embodiment 1.

FIG. 9 is a flow chart representing a second example of the flow of a process performed by the modifying unit included in the biological information estimating device in accordance with Embodiment 1.

### DESCRIPTION OF EMBODIMENTS

The following will describe an embodiment of the present disclosure with reference to drawings. Identical and equivalent elements in the drawings are denoted by the same reference numerals, and description thereof is not repeated.

### 1 Embodiment 1

### 1.1 Biological Information Estimating Device

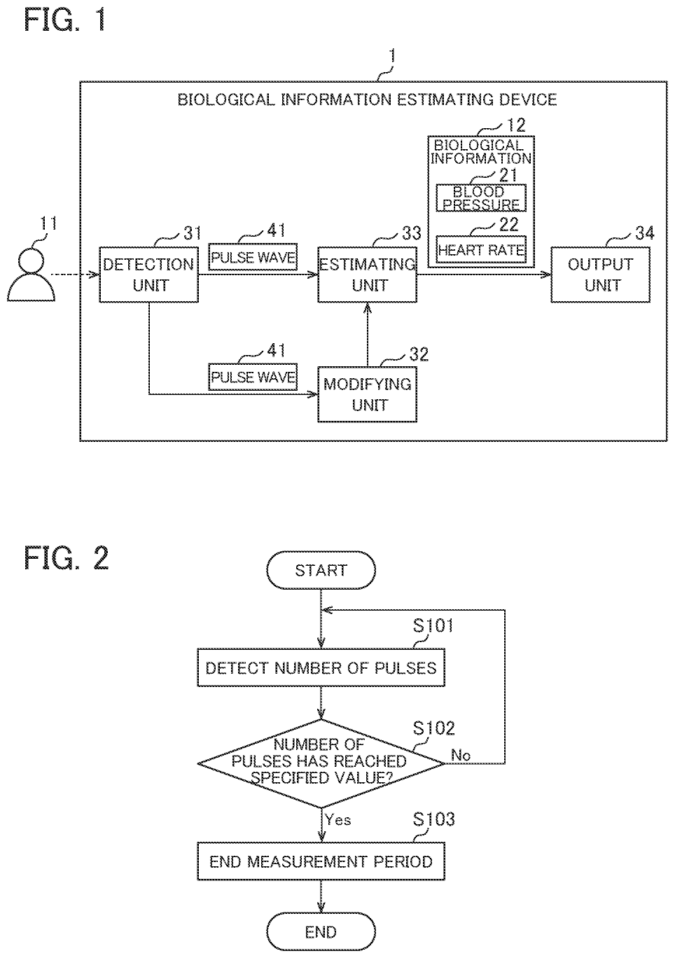

FIG. 1 is a block diagram of a biological information estimating device in accordance with Embodiment 1.

A biological information estimating device 1 shown in FIG. 1 estimates biological information 12 on a living organism 11. The living organism 11 on which the biological information 12 is estimated is a human. The living organism 11 may be a non-human animal. The estimated biological information 12 represents a condition of the living organism 11. The biological information 12 includes a blood pressure 21 and a pulse rate 22. The biological information 12 may include biological information other than the blood pressure 21 and the pulse rate 22.

Referring to FIG. 1, the biological information estimating device 1 includes a detection unit 31, a modifying unit 32, an estimating unit 33, and an output unit 34.

The detection unit 31 detects a pulse wave 41 on the living organism 11.

The modifying unit 32 modifies the length of a measurement period on the basis of the pulse pace of the living organism 11. This configuration enables adjusting the length of a measurement time suitably to the pulse pace of the living organism 11. The modifying unit 32 utilizes the fact that the rate of cyclic temporal changes of the pulse wave 41 reflects the pulse pace, to modify the length of the measurement period on the basis of the rate of cyclic temporal changes of the pulse wave 41. The modifying unit 32 reduces the length of the measurement period with an increase in the pulse pace. This configuration enables reducing the time required to estimate the biological information 12 on the living organism 11 without having to identify the living organism 11. For instance, the time is reduced to or below 10 seconds.

The estimating unit 33 estimates the biological information 12 from the pulse wave 41 detected in the measurement period.

The output unit 34 outputs the estimated biological information 12. The output unit 34 includes, for example, a display device that displays a screen showing the biological information 12, a speaker that projects sound representing the biological information 12; and/or a transmission circuit that transmits a signal representing the biological information 12.

### 1.2 Detection Unit

The detection unit 31 detects the pulse wave 41 at a body part of the living organism 11. The body part where the pulse wave 41 is detected is the fingertip, the palm, the bottom of the foot, the cheek, the forehead, the nose, or the jaw. That body part may be any body part other than the fingertip, the palm, the bottom of the foot, the cheek, the forehead, the nose, and the jaw. The detection unit 31 may either detect the pulse wave 41 at one of these body parts or detect the pulse wave 41 simultaneously at some of the body parts. These plural body parts may be, as an example, a combination of a fingertip of the left hand and a fingertip of the right hand, a combination of two or more body parts selected from, for example, the cheek, the forehead, and the nose, which are all parts on the face, or a combination of a fingertip and the face. The detection unit 31 preferably detects the pulse wave 41 in real time.

In a first example of the present disclosure, the detection unit 31 includes a contact sensor.

The contact sensor is brought into contact with a fingertip of the living organism 11 to detect the pulse wave 41 at the fingertip that is in contact with the contact sensor.

The contact sensor includes a light-emitting unit and a light-receiving unit.

The light-emitting unit emits light. The emitted light may be either visible light or invisible light. The visible light is, for example, red light or green light. The invisible light is, for example, infrared light. The light-emitting unit includes, for example, a light-emitting diode (LED).

The light-receiving unit receives the light generated by the reflection and diffusion by the fingertip that is in contact with the detection unit 31 of the light emitted by the light-emitting unit and outputs a signal in accordance with the amount of the received light. Temporal changes of the amplitude of the outputted signal form the pulse wave 41 to be detected. This amount of light reflects the volume of the blood flowing in blood vessels inside the fingertip. The pulse wave 41 to be detected therefore reflects temporal changes in the volume of the blood. The light-receiving unit includes, for example, a photodiode.

The detection unit 31 may include a signal processing unit that performs signal processing on the detected pulse wave 41 to remove noise components from the pulse wave 41. The signal processing performed includes, for example, a process of removing long-period, low-frequency noise components and a process of removing short-period, high-frequency noise components. The former process is, for example, highpass filtering or trend removal. The latter process is, for example, lowpass filtering. The processing performed may be either an analog signal process implemented by an electronic circuit or a digital signal process implemented by a processor.

In a second example of the present disclosure, the detection unit 31 includes an image capturing unit and an image processing unit.

The image capturing unit captures an image of the living organism 11 to obtain an image thereof. The image capturing unit includes, for example, an RGB camera.

The image processing unit determines whether or not the obtained image contains the living organism 11, and upon determining that the image contains the living organism 11, outputs pixel values of the pixels at which the living organism 11 is imaged. Temporal changes of the outputted pixel values are the pulse wave 41 to be detected. The image processing unit includes, for example, a central processing unit (CPU) that executes programs. The processing performed by the image processing unit may be entirely or partially implemented by a dedicated electronic circuit.

When detecting the pulse wave 41 simultaneously at a fingertip of the left hand and at a fingertip of the right hand, the detection unit 31 includes a contact sensor for the left-hand fingertip and a contact sensor for the right-hand fingertip. The left-hand fingertip contact sensor is brought into contact with a fingertip of the left hand to detect the pulse wave 41 at the fingertip of the left hand that is in contact with the left-hand fingertip contact sensor. The right-hand fingertip contact sensor is brought into contact with a fingertip of the right hand to detect the pulse wave 41 at the fingertip of the right hand that is in contact with the right-hand fingertip contact sensor.

When the detection unit 31 detects the pulse wave 41 simultaneously at two or more body parts on the face, the image capturing unit captures an image of the face of the living organism 11 to obtain an image of the face. Additionally, the image processing unit computes the pulse wave 41 from the pixel values of the pixels at which the two or more body parts are imaged.

When the detection unit 31 detects the pulse wave 41 simultaneously at a fingertip and on the face, the contact sensor is brought into contact with the fingertip of the living organism 11 to detect the pulse wave 41 at the fingertip that is in contact with the contact sensor. Additionally, the image capturing unit captures an image of the face of the living organism 11 to obtain an image of the face. Additionally, the image processing unit computes the pulse wave 41 from the pixel values of the pixels at which the face is imaged.

### 1.3 Modifying Unit

The modifying unit 32 detects the number of pulses in the detected pulse wave 41 after the measurement period is started and ends the measurement period when the detected number of pulses has reached a specified value. The modifying unit 32 includes, for example, a CPU for executing programs. The processing performed by the modifying unit 32 may be entirely or partially implemented by a dedicated electronic circuit.

The detected pulse wave 41 represents temporal changes of the detection amount detected on the living organism 11. Temporal changes of the pulse wave 41 contains cyclic temporal changes that result from cyclic heartbeats of the heart of the living organism 11. A pulse in the pulse wave 41 represents temporal changes of the detection amount, extracted from the pulse wave 41, in a period having a length of time taken by the heart of the living organism 11 to make one heartbeat. Both ends of the pulse may be either a local minimum point where the detection amount reaches a local minimum or a local maximum point where the detection amount reaches a local maximum. Therefore, both ends of the pulse may be either a trough or a peak on the waveform of the pulse wave 41.

Even if the number of pulses in the pulse wave 41 detected in the measurement period is 1, the estimating unit 33 is still capable of estimating the biological information 12 from this single pulse. Therefore, the specified value may be equal to 1. However, the estimation of the biological information 12 by the estimating unit 33 becomes increasingly precise with a larger number of pulses. Therefore, the specified value is preferably greater than or equal to 2 and more preferably greater than or equal to 5.

### 1.4 Processing Performed by Modifying Unit

FIG. 2 is a flow chart representing a first example of the flow of a process performed by the modifying unit included in a biological information estimating device in accordance with Embodiment 1.

The modifying unit 32 performs steps S101 to S103 shown in FIG. 2.

In step S101, the modifying unit 32 detects the number of pulses contained in the pulse wave 41 detected after the measurement period is started.

In subsequent step S102, the modifying unit 32 determines whether or not the detected number of pulses has reached the specified value. If it is determined that the detected number of pulses has reached the specified value, step S103 is performed. If it is determined that the detected number of pulses has not reached the specified value, step S101 is performed again.

In step S103, the modifying unit 32 ends the measurement period.

In steps S101 to S103, the modifying unit 32 does not end the measurement period and continues the measurement period until the number of pulses contained in the pulse wave 41 detected after the measurement period is started reaches the specified value, and ends the measurement period when the number of those pulses has reached the specified value.

### 1.5 Changes in Length of Measurement Period with Pulse Pace

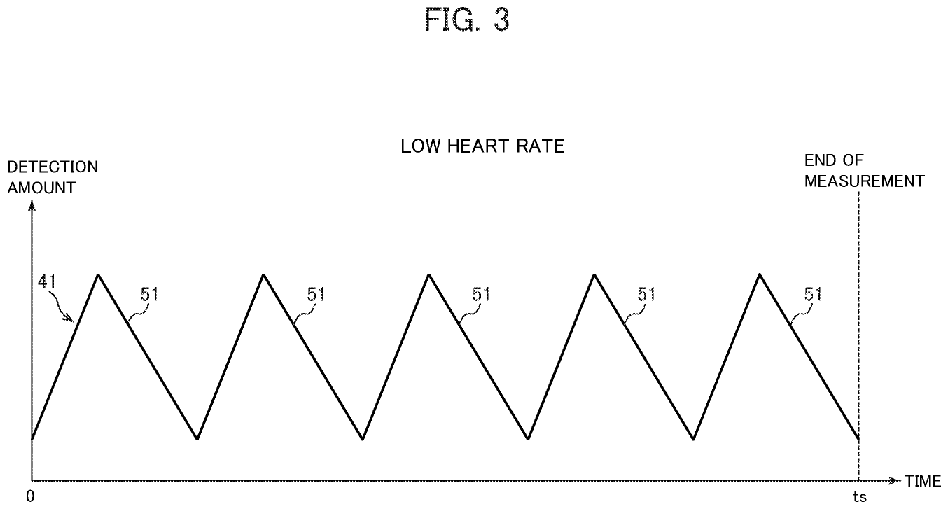

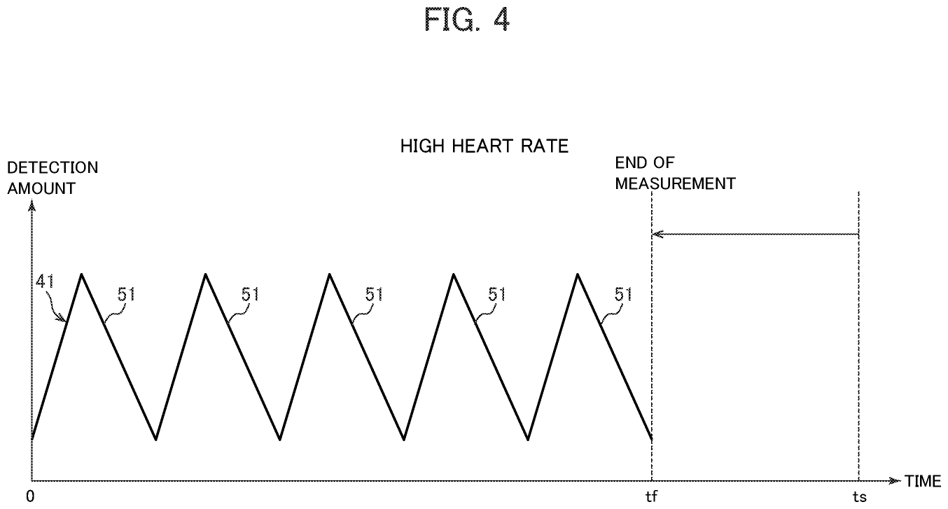

FIG. 3 is a graph representing an exemplary timing for the modifying unit included in the biological information estimating device in accordance with Embodiment 1 to end the measurement period when the pulse pace of the living organism is slow. FIG. 4 is a graph representing an exemplary timing for the modifying unit included in the biological information estimating device in accordance with Embodiment 1 to end the measurement period when the pulse pace of the living organism is fast.

In FIGS. 3 and 4, time is plotted on the horizontal axis. In addition, the detection amount detected on the living organism 11 is plotted on the vertical axis.

In the examples shown in FIGS. 3 and 4, the modifying unit 32 detects the number of pulses 51 contained in the pulse wave 41 detected after the measurement period is started at time 0, and ends the measurement period when the number of the detected pulses 51 has reached 5. Therefore, the modifying unit 32 ends the measurement period when the time equal to 5 times the period of the pulse wave 41 has elapsed after time 0. Therefore, the modifying unit 32 ends the measurement period at time ts, which is relatively late, as shown in FIG. 3, when the pulse pace of the living organism 11 is slow with the pulse wave 41 having a long period. On the other hand, the modifying unit 32 ends the measurement period at time tf, which is relatively early, as shown in FIG. 4, when the pulse pace of the living organism 11 is fast with the pulse wave 41 having a short period. The estimating unit 33 estimates the biological information 12 from five of the pulses 51 detected in the measurement period.

The modifying unit 32 detects the number of pulses contained in the pulse wave 41 by performing signal processing on the pulse wave 41. The signal processing performed includes, for example, peak detection and frequency analysis.

### 1.6 Detecting Number of Pulses by Means of Peak Detection

To detect the number of the pulses 51 by performing peak detection on the pulse wave 41, the modifying unit 32 extracts local maximum points from the pulse wave 41 and specifies a portion of the pulse wave 41 that is present between adjacent local maximum points as one of the pulses 51. Alternatively, the modifying unit 32 extracts local minimum points from the pulse wave 41 and specifies a portion of the pulse wave 41 that is present between adjacent local minimum points as one of the pulses 51.

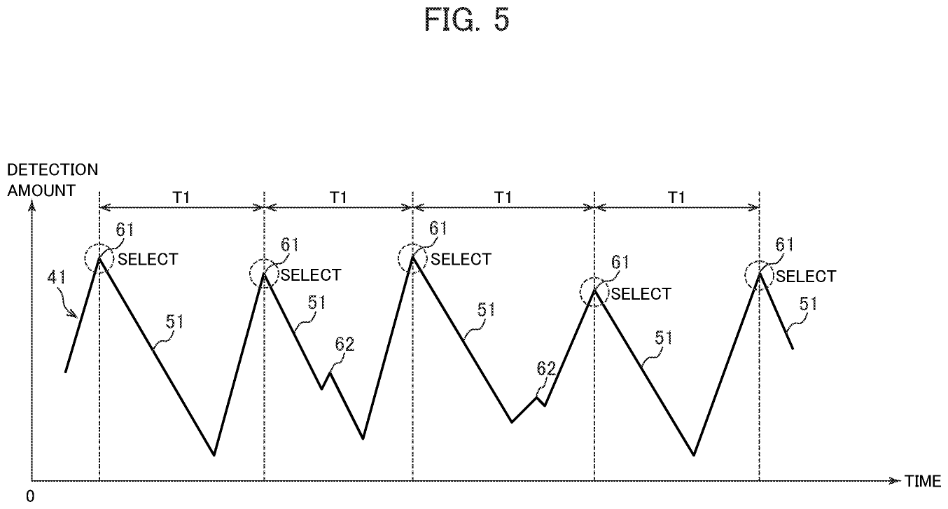

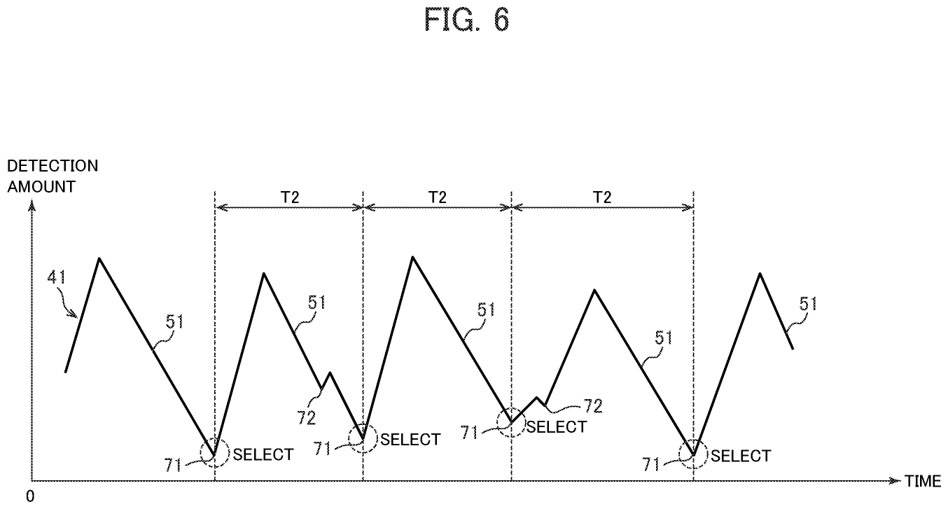

FIG. 5 is a graph representing a first example of local maximum point selection performed by the modifying unit included in the biological information estimating device in accordance with Embodiment 1. FIG. 6 is a graph representing a first example of local minimum point selection performed by the modifying unit included in the biological information estimating device in accordance with Embodiment 1.

In FIGS. 5 and 6, time is plotted on the horizontal axis. In addition, the detection amount detected on the living organism 11 is plotted on the vertical axis.

In the first example, the modifying unit 32 selects, in descending order of height, a plurality of local maximum points 61 on the pulse wave 41 detected after the start of the measurement period in such a manner that each time interval T1 between adjacent local maximum points 61 falls within a specified range, and specifies a portion of the pulse wave 41 that is present between adjacent local maximum points 61 as one of the pulses 51, as shown in FIG. 5. The range is specified on the basis of a typical pulse rate which is 50 to 90 beats/minute. This configuration enables selecting overall local maximum points 61 that can each serve as a suitable separation between the two adjacent pulses 51. The configuration also enables restraining selection of regional local maximum points 62 that cannot serve as a suitable separation. Alternatively, as shown in FIG. 6, the modifying unit 32 selects, in descending order of depth, a plurality of local minimum points 71 on the pulse wave 41 detected after the start of the measurement period in such a manner that each time interval T2 between adjacent local minimum points 71 falls within a specified range, and specifies a portion of the pulse wave 41 that is present between adjacent local minimum points 71 as one of the pulses 51. The range is specified on the basis of a typical pulse rate, which is 50 to 90 beats/minute. This configuration enables selecting overall local minimum points 71 that can each serve as a suitable separation between the two adjacent pulses 51. The configuration also enables restraining selection of regional local minimum points 72 that cannot serve as a suitable separation.

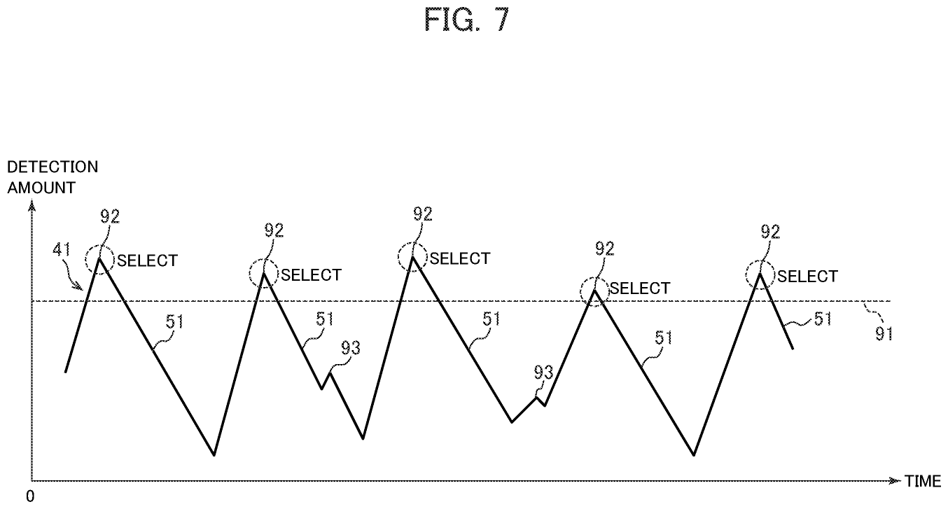

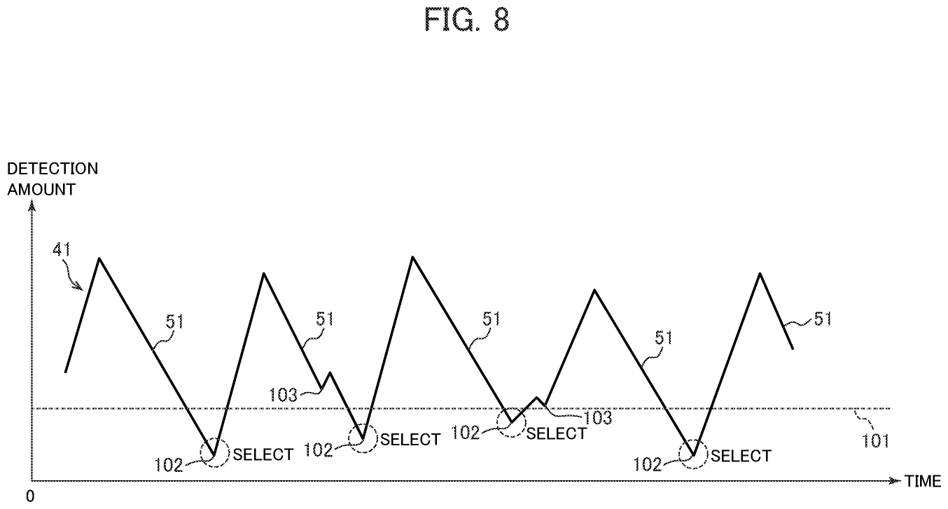

FIG. 7 is a graph representing a second example of local maximum point selection performed by the modifying unit included in the biological information estimating device in accordance with Embodiment 1. FIG. 8 is a graph representing a second example of local minimum point selection performed by the modifying unit included in the biological information estimating device in accordance with Embodiment 1.

In FIGS. 7 and 8, time is plotted on the horizontal axis. In addition, the detection amount detected on the living organism 11 is plotted on the vertical axis.

In the second example, the modifying unit 32 selects a plurality of local maximum points 92 that have a height greater than a height 91 specified based on the pulse wave 41 detected after the start of the measurement period and specifies a portion of the pulse wave 41 that is present between adjacent local maximum points 92 as one of the pulses 51, as shown in FIG. 7. The height 91 is specified on the basis of the amplitude of the pulse wave 41. This configuration enables selecting overall local maximum points 92 that can each serve as a suitable separation between the two adjacent pulses 51. The configuration also enables restraining selection of regional local maximum points 93 that cannot serve as a suitable separation. Alternatively, as shown in FIG. 8, the modifying unit 32 selects a plurality of local minimum points 102 that have a depth greater than a depth 101 specified based on the pulse wave 41 detected after the start of the measurement period and specifies a portion of the pulse wave 41 that is present between adjacent local minimum points 102 as one of the pulses 51. The depth 101 is specified on the basis of the amplitude of the pulse wave 41. This configuration enables selecting overall local minimum points 102 that can each serve as a suitable separation between the two adjacent pulses 51. The configuration also enables restraining selection of regional local minimum points 103 that cannot serve as a suitable separation.

### 1.7 Detecting Number of Pulses by Means of Frequency Analysis

To detect the number of the pulses 51 by performing frequency analysis on the pulse wave 41, the modifying unit 32 detects the period of the pulse wave 41 and divides the time at which the pulse wave 41 is detected, that is, the time that has elapsed since the start of the reception period, by the detected period, to detect the number of the pulses 51. The decimal digits in the result of division of the time at which the pulse wave 41 is detected by the detected period may be rounded down so that the number of the pulses 51 can be an integer. For instance, when the time at which the pulse wave 41 is detected is 3 seconds, and the detected period is 0.9 seconds, the decimal digits in the result (3/0.9=3.333 . . . ) of division of the time at which the pulse wave 41 is detected by the detected period may be rounded down, so that the number of the pulses 51 can be 3.

The modifying unit 32 detects the period of the pulse wave 41 by performing, for example, fast Fourier transform (FFT) or maximum entropy (MEM) on the pulse wave 41.

### 1.8 Determining Length of Measurement Period Based on Frequency

The modifying unit 32 may detect the frequency of the pulse wave 41 detected after the start of the measurement period and reduce the length of the measurement period with an increase in the detected frequency.

The modifying unit 32 detects the frequency of the pulse wave 41 by performing, for example, FFT or MEM on the pulse wave 41.

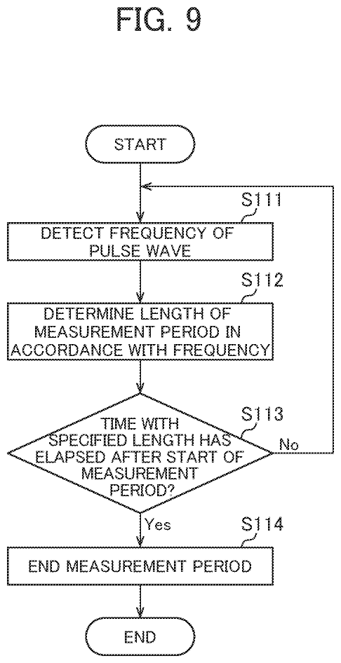

FIG. 9 is a flow chart representing a second example of the flow of a process performed by the modifying unit included in the biological information estimating device in accordance with Embodiment 1.

To detect the frequency of the pulse wave 41 and reduce the length of the measurement period with an increase in the detected frequency, the modifying unit 32 performs steps S111 to S114 shown in FIG. 9.

In step S111, the modifying unit 32 detects the frequency of the pulse wave 41 detected after the start of the measurement period.

In subsequent step S112, the modifying unit 32 determines the length of the measurement period in accordance with the detected frequency. The modifying unit 32 reduces the length of the measurement period with an increase in the detected frequency. For instance, the modifying unit 32 determines the length of the measurement time by multiplying the reciprocal of the detected frequency by a coefficient. This multiplication coefficient is greater than or equal to 1, preferably greater than or equal to 2, and more preferably greater than or equal to 5.

In subsequent step S113, the modifying unit 32 determines whether or not time that has a specified length has elapsed since the start of the measurement period. If it is determined that this time has elapsed, step S114 is performed. If it is determined that the time has not elapsed, step S111 is performed again.

In step S114, the modifying unit 32 ends the measurement period.

In steps S111 to S114, the modifying unit 32 does not end the measurement period and continues the measurement period until the time that has elapsed after the start of the measurement period becomes sufficiently long, and ends the measurement period when the time has become sufficiently long.

### 1.9 Estimating Unit

The estimating unit 33 estimates the blood pressure 21 from the waveform and/or frequency dependency of the pulse wave 41 detected in the measurement period. When the detection unit 31 detects the pulse wave 41 simultaneously at two or more body parts, the estimating unit 33 may estimate the blood pressure 21 from the correlation of the pulse waves 41 detected simultaneously at the two or more body parts. This configuration enables the biological information estimating device 1 to estimate the biological information 12 without a need to use a cuff.

The estimating unit 33 also estimates the pulse rate 22 from the number of the pulses 51 contained in the pulse wave 41 detected in the measurement period. For instance, the estimating unit 33 estimates the pulse rate 22 by dividing the number of the pulses 51 by the length of the measurement period.

The estimating unit 33 is, for example, a CPU for executing programs. The processing performed by the modifying unit 32 may be entirely or partially implemented by a dedicated electronic circuit.

### 1.10 Comparison with Cuff-Type Electronic Blood Pressure Meter

A cuff-type electronic blood pressure meter changes pressure inside the cuff. In addition, the cuff itself serves as a sensor so that the cuff can detect a pulse wave. In addition, the blood pressure is estimated from the detected pulse wave. When the blood pressure is detected by oscillometry, pressure is changed in the cuff wound around the arm. In addition, the blood pressure is estimated from the amplitude of the detected pulse wave. To estimate the blood pressure, the pulse wave must be detected over a plurality of cycles while the pressure in the cuff is gradually changed. The measurement time can be reduced by changing the pressure in the cuff at a maximum rate at which as many heartbeats as needed to estimate the blood pressure can be detected. However, this maximum rate can be calculated only when the blood pressure and pulse rate of the user are known. Therefore, it is impossible to reduce the measurement time when the blood pressure of the user who uses the electronic blood pressure meter for the first time is to be estimated. In addition, to reduce the measurement time, it is required to record previous blood pressure and pulse rate estimates for each user. In addition, it is also required to determine whether or not the user has previously used the electronic blood pressure meter, and it is necessary to determine which one of the users who previously used the electronic blood pressure meter the user is.

In contrast, in the biological information estimating device 1, the information used to estimate the blood pressure 21 and the pulse rate 22 and the information used to determine the length of the measurement period are obtained from the pulse wave 41 detected after the start of the measurement period. Therefore, the biological information estimating device 1 does not need to obtain these sets of information before the start of the measurement period. Therefore, time and labor to obtain information on the living organism 11 in advance can be eliminated.

The present disclosure is not limited to the description of the embodiments and examples above. Any structure detailed in the embodiments and examples may be replaced by a practically identical structure, a structure that achieves the same effect and function, or a structure that achieves the same purpose.

What is claimed is:

1. A biological information estimating device comprising:
a detection unit configured to detect a pulse wave on a living organism;
a modifying unit configured to modify a length of a measurement period based on a pulse pace of the living organism; and
an estimating unit configured to estimate biological information from the pulse wave detected in the measurement period.

2. The biological information estimating device according to claim 1, wherein the modifying unit reduces the length with an increase in the pulse pace.

3. The biological information estimating device according to claim 1, wherein the modifying unit ends the measurement period when a count of pulses contained in the pulse wave detected after a start of the measurement period has reached a specified value.

4. The biological information estimating device according to claim 3, wherein the modifying unit selects a plurality of local maximum points in descending order of height from the pulse wave detected after the start of the measurement period in such a manner that a time interval between adjacent local maximum points falls in a specified range, and specifies a portion of the pulse wave detected after the start of the measurement period as a single pulse, the portion being present between the adjacent local maximum points.

5. The biological information estimating device according to claim 3, wherein the modifying unit selects a plurality of local minimum points in descending order of depth from the pulse wave detected after the start of the measurement period in such a manner that a time interval between adjacent local minimum points falls in a specified range, and specifies a portion of the pulse wave detected after the start of the measurement period as a single pulse, the portion being present between the adjacent local minimum points.

6. The biological information estimating device according to claim 3, wherein the modifying unit selects a plurality of local maximum points that have a height greater than or equal to a specified height from the pulse wave detected after the start of the measurement period and specifies a portion of the pulse wave detected after the start of the measurement period as a single pulse, the portion being present between adjacent local maximum points.

7. The biological information estimating device according to claim 3, wherein the modifying unit selects a plurality of local minimum points that have a depth greater than or equal to a specified depth from the pulse wave detected after the start of the measurement period and specifies a portion of the pulse wave detected after the start of the measurement period as a single pulse, the portion being present between adjacent local minimum points.

8. The biological information estimating device according to claim 1, wherein the modifying unit reduces the length with an increase in frequency of the pulse wave detected after the start of the measurement period.

9. The biological information estimating device according to claim 1, wherein the biological information includes blood pressure.

10. The biological information estimating device according to claim 1, wherein the biological information includes a pulse rate.

11. A biological information estimating method comprising:
a) detecting a pulse wave on a living organism;
b) modifying a length of a measurement period based on a pulse pace of the living organism; and
c) estimating biological information from the pulse wave detected in the measurement period.

12. The biological information estimating device according to claim 2, wherein the modifying unit ends the measurement period when a count of pulses contained in the pulse wave detected after a start of the measurement period has reached a specified value.

13. The biological information estimating device according to claim 12, wherein the modifying unit selects a plurality of local maximum points in descending order of height from the pulse wave detected after the start of the measurement period in such a manner that a time interval between adjacent local maximum points falls in a specified range, and specifies a portion of the pulse wave detected after the start of the measurement period as a single pulse, the portion being present between the adjacent local maximum points.

14. The biological information estimating device according to claim 12, wherein the modifying unit selects a plurality of local minimum points in descending order of depth from the pulse wave detected after the start of the measurement period in such a manner that a time interval between adjacent local minimum points falls in a specified range, and specifies a portion of the pulse wave detected after the start of the measurement period as a single pulse, the portion being present between the adjacent local minimum points.

15. The biological information estimating device according to claim 12, wherein the modifying unit selects a plurality of local maximum points that have a height greater than or equal to a specified height from the pulse wave detected after the start of the measurement period and specifies a portion of the pulse wave detected after the start of the measurement period as a single pulse, the portion being present between adjacent local maximum points.

16. The biological information estimating device according to claim 12, wherein the modifying unit selects a plurality of local minimum points that have a depth greater than or equal to a specified depth from the pulse wave detected after the start of the measurement period and specifies a portion of the pulse wave detected after the start of the measurement period as a single pulse, the portion being present between adjacent local minimum points.

17. The biological information estimating device according to claim 2, wherein the modifying unit reduces the length with an increase in frequency of the pulse wave detected after the start of the measurement period.
